(19) United States
(12) Patent Application Publication
(10) Pub. No.: US 2009/0232913 A1
(43) Pub. Date: Sep. 17, 2009

(54) Composition for the Control of Cholesterol Levels

(76) Inventors: Momir Mikov, Dunedin (NZ); Robert Syndecombe Bower, Dunedin (NZ)

Correspondence Address: FROMMER LAWRENCE & HAUG, 745 FIFTH AVENUE- 10TH FL., NEW YORK, NY, 10151, US

(21) Appl. No.: 11/913,358

(22) PCT Filed: May 2, 2006
(86) PCT No.: PCT/AU2006/000579

§ 371 Date: Nov. 24, 2008

(30) Foreign Application Priority Data
May 2, 2005 (AU) 2005902196

(51) Int. Cl. A61K 36/28; A61K 31/704; A61P 9/00

(52) U.S. Cl. 424/725; 514/34

(57) ABSTRACT

The present invention relates to a composition used to control cholesterol levels. In particular, the present invention relates to a method for the treatment or prevention of disorders or diseases mediated by hyperlipidemia, said method comprising administering to a mammalian subject in need thereof an effective amount of a composition comprising an extract from at least one plant from the genus Stevia and at least one bile salt, salt thereof or derivative thereof admixed in a form suitable for therapeutic administration.

### FIELD

The present invention relates to a composition used to control cholesterol levels. In particular, the present invention relates to a composition comprising an extract from at least one plant of the genus Stevia and at least one bile salt.

### BACKGROUND

Atherosclerosis is the leading cause of death in a number of first world countries like the United States. Atherosclerosis is the formation of plaques in arterial walls that can occlude the vessel lumen and obstruct blood flow through the vessel. Morbidity and mortality generally occur through end organ damage and organ dysfunction resulting from ischemia. The most common forms of ischemia that end in organ damage are myocardial infarction and cerebrovascular accidents. Disability or death often results from these vascular events.

Arteriosclerotic lesions are plaques that form by accumulation of cholesterol, cholesterol esters, and phospholipids and proliferation of smooth muscle cells in the intima of major arteries. Lipid contributes a major portion of the plaque volume (generally 30-65% dry weight) see, for example, Small, 1988, Arteriosclerosis, 8:103-129. In fact, the risk of developing arteriosclerosis is directly related to the concentration of certain forms of plasma cholesterol. Lipids, including cholesterol, are generally insoluble in aqueous plasma. Plasma lipids are carried by soluble lipoprotein complexes. These lipoprotein complexes consist of an inner core of non-polar lipids (cholesteryl esters and triglycerides) and a surface layer of hydrophilic proteins and polar lipids (phospholipids and non-esterified cholesterol). Different proteins are present in the surface coat of different lipoprotein complexes (lipoproteins). The different lipoproteins perform different functions in lipid metabolism.

Five classes of lipoproteins are known. Some lipoproteins carry triglycerides and cholesterol from the liver to peripheral tissues while others transport lipids to the liver. Cholesterol may be metabolized in the liver to bile salts that are excreted, thus lowering total body cholesterol. Two lipoproteins, low density lipoproteins (LDL) and high density lipoproteins (HDL), have a high degree of association with the development of atherosclerosis. LDL has a high cholesterol concentration, delivers lipids to cells of peripheral tissues, and is associated with a high risk of atherosclerosis. HDL also has a relatively high cholesterol concentration, but carries lipids to the liver for metabolism into bile salts and is associated with decreasing the risk of developing atherosclerosis. Consequently, it is appreciated by most medical practitioners that in order to avoid the development of atherosclerosis it is preferable for individuals to have low total cholesterol levels (less than 200 mg/dL), high HDL levels (greater than 70 mg/dL in males & 80 mg/dL in females) and low LDL levels (less than 130 mg/dL).

Presently, the most effective treatment of atherosclerosis is prevention. There is evidence that the progression and accumulation of lipids in lesions can be halted when plasma LDL concentrations are kept to near normal levels (Reynolds, 1989, Circulation, 79:1146-1148). Current preventive management of atherosclerotic disease has focused on the use of drugs in conjunction with dietary restrictions to regulate plasma cholesterol levels. Moreover, antioxidant therapies which suppress the formation and uptake of modified LDL particles by the cells of the arterial wall are also proving beneficial (Chisolm, 1991, Clin. Cardiol, 14:25-30).

However, while hypocholesterolemic drugs induce favourable plasma cholesterol changes which appear to slow the progression of atherosclerosis, they do hot generally induce conditions that promote the efflux and removal of cholesterol. What is needed in the art is a medical treatment or prevention for atherosclerosis that not only will slow progression of lesions, but also predictably cause regression and shrinkage of established plaques.

### SUMMARY OF THE INVENTION

The present inventors have shown that extracts of Stevia plants admixed with bile salts is effective in controlling hyperlipidemia and thereby atherosclerosis. Accordingly, in a first aspect, the present invention provides a composition comprising an extract from at least one plant from the genus Stevia and at least one bile salt, salt thereof or derivative thereof admixed in a form suitable for therapeutic administration.

It will be appreciated by those skilled in the art that the extract may be obtained from any plant of the genus Stevia. Preferably, the extract is obtained from a plant selected from the group consisting of Stevia lemmonii, Stevia micrantha, Stevia ovata, Stevia plummerae, Stevia rebaudiana, Stevia salicifolia, Stevia serrata and Stevia viscida. Most preferably, the extract is obtained from Stevia rebaudiana.

The extract from the Stevia plant can comprise a number of compounds including apigenin, austroinulin, avicularin, beta-sitosterol, caffeic acid, campesterol, caryophyllene, centaureidin, chlorogenic acid, chlorophyll, cosmosiin, cynaroside, daucosterol, diterpene glycosides, dulcosides A-B, foeniculin, formic acid, gibberellic acid, gibberellin, indole-3-acetonitrile, isoquercitrin, isosteviol, jhanol, kaempferol, kaurene, lupeol, luteolin, polystachoside, quercetin, quercitrin, rebaudioside A-F, scopoletin, sterebin A-H, steviol, steviolbioside, steviolmonoside, stevioside, stevioside α-3, stigmasterol, umbelliferone, and xanthophylls. Preferably, the extract comprises at least one of steviolbioside, steviolmonoside, stevioside, stevioside α-3 and stigmasterol. Most preferably, the extract comprises at least stevioside.

The extract from the Stevia plant is admixed with at least one bile salt, salt thereof or bile salt derivative. There are many known bile salts including chenodeoxycholate, cholate, deoxycholate, fusidate, glycholate, glycochenodeoxycholate, glycocholate, glycodeoxycholate, glycolithocholate, glycoursodeoxycholate, lithocholate, taurochenodeoxycholate, taurocholate, taurodeoxycholate, taurodihydrofusidate, taurolithocholate, taurouodeoxycholate and ursodeoxycholate. In one embodiment, the bile salts are salts of chyodoxycholic acid (eg 3α,6α-Dihydroxy-5β-cholanic acid); chiocholic acid (eg 3α,6α,7α-Trihydroxy-5β-cholanic acid); and ursodeoxicholic acid (eg 3α,7β-Dihydroxy-5β-cholanic acid, 3α,7α-Dihydroxy-24-ethyl-5β-cholestan-26-carboxylic acid, 2β,3α,7α,12α-Tetrahydroxy-5β-cholestan-24-carboxylic acid, 1β, 3α,7α,12α-Tetrahydroxy-5β-cholestan-24-carboxylic acid, 3α,7β,12α-Trihydroxy-5β-cholestan-26-carboxylic acid, 3α,7α-Dihydroxy-7-keto-5β-cholestan-26-carboxylic acid, 3α,7α-Dihidroksy-5β-cholestan-26-carboxylic acid, 3α,12α,22-Trihydroxy-5β-cholestan-26-karboxylic acid, 3α,7α,12α,26-Tetrahydroxy-5β-cholestan-23-en-27-carboxylic acid, 3α,7α,12α,24-Tetrahydroxy-24-methyl-5β-cholestan-26-carboxylic acid, 3α,7α,12α-Trihydroxy-26,27-dinor-5β-cholestan-25-carboxylic acid).

In some embodiments, the composition of the present invention is also admixed with suitable pharmaceutical carriers, diluents and/or vehicles. Suitable carriers include one or more substances which may also act as flavouring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pharmaceutically accepted oils, or a mixture of both. The liquid can also contain other suitable pharmaceutical additions such as buffers, preservatives, flavouring agents, viscosity or osmo-regulators, stabilizers or suspending agents. Examples of suitable liquid carriers include water with or without various additives, including carboxypolymethylene as a pH-regulated gel.

It will be appreciated by those skilled in the art, that the compositions of the present invention can be used to treat or prevent hyperlipidemia in a mammalian subject. Accordingly, in a second aspect the present invention provides a method for the treatment or prevention of hyperlipidemia in a mammalian subject comprising the administration of a therapeutically effective amount of a composition comprising an extract from at least one plant from the genus Stevia and at least one bile salt, salt thereof or derivative thereof.

The mammalian subject may be any mammal suffering from or prone to hyperglycaemia. Preferably, the mammalian subject is a dog, a cat, a livestock animal, a horse, or primate including a human. More preferably the mammalian subject is a human.

The hyperlipidemia suffered by the mammalian subject can result from any known cause.

Accordingly, in a third aspect the present invention provides a method for the treatment of disorders or diseases mediated by hyperlipidemia, said method comprising administering to a mammalian subject in need thereof an effective amount of a composition comprising an extract from at least one plant from the genus Stevia and at least one bile salt, salt thereof or derivative thereof admixed in a form suitable for therapeutic administration.

In a fourth aspect the present invention provides, a method for lowering blood cholesterol in a mammalian subject, said method comprising administering to a mammalian subject in need thereof an effective amount of a composition comprising an extract from at least one plant from the genus Stevia and at least one bile salt, salt thereof or derivative thereof admixed in a form suitable for therapeutic administration.

In a seventh aspect, the present invention provides a method for treating or preventing atherosclerosis, hypercholesterolemia, hypertriglyceridemia, hyperlipidemia, tissue ischemia or myocardial ischemia in a mammalian subject, the method comprising administering to said mammal a composition comprising an extract from at least one plant from the genus Stevia and at least one bile salt, salt thereof or derivative thereof admixed in a form suitable for therapeutic administration.

In an eighth aspect, the present invention provides a method for treating or preventing atherosclerosis, hypercholesterolemia, hypertriglyceridemia, hyperlipidemia, tissue ischemia or myocardial ischemia in a mammalian subject, the method comprising administering to said mammal a composition comprising an extract from at least one plant from the genus Stevia and at least one bile salt, salt thereof or derivative thereof in combination with at least one additional compound useful for the treatment or prevention of atherosclerosis, hypercholesterolemia, hypertriglyceridemia, hyperlipidemia, tissue ischemia or myocardial ischemia.

Administration of the compositions of the present invention includes any route routinely used for the administration of pharmaceutical agents. Preferably, the route of administration is selected from the group consisting of oral, rectal, parenteral (subcutaneous, intramuscular, intravenous) and transdermal. More preferably, the route of administration is oral administration.

### DETAILED DESCRIPTION OF THE PREFERRED EMBODIMENTS

Before the present methods are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “% a,” “an,” and “the” include plural reference unless the context clearly dictates otherwise. Thus, for example, a reference to “an extract” is a reference to one or more extracts and equivalents thereof known to those skilled in the art, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All publications mentioned herein are cited for the purpose of describing and disclosing the methods, protocols and reagents which are reported in the publications and which might be used in connection with the invention. Nothing herein is to be construed as an admission that the invention is not entitled to antedate such disclosure by virtue of prior invention.

In its broadest aspect the present invention relates to a composition comprising an extract from at least one plant of the genus Stevia and at least one bile salt, salt thereof or derivative thereof.

The terms “plant of the genus Stevia” or “Stevia plants” are used herein interchangeably. Plants of the genus Stevia are perennial shrubs that grow up to about 1 m tall and have leaves 2-3 cm long. These plants belong to the Aster family and are indigenous to the northern regions of South America. The genus Stevia contains at least 7 species and 10 accepted taxa including Stevia lemmonii, Stevia micrantha, Stevia ovata, Stevia plummerae, Stevia rebaudiana, Stevia salicifolia, Stevia serrata and Stevia viscida.

In addition to being a sweetener, Stevia plants are considered in Brazilian herbal medicine to be useful as a hypoglycaemic agent, hypotensive agent, diuretic agent and a cardiotonic agent. The leaf is used for treatment of diabetes, obesity, cavities, hypertension, fatigue, depression, sweet cravings, and infections.

Over 100 phytochemicals have been discovered in plants of the genus Stevia to date. These plants are rich in terpenes and flavonoids. The main plant chemicals in Stevia plants include: apigenin, austroinulin, avicularin, beta-sitosterol, caffeic acid, campesterol, caryophyllene, centaureidin, chlorogenic acid, chlorophyll, cosmosiin, cynaroside, daucosterol, diterpene glycosides, dulcosides A-B, foeniculin, formic acid, gibberellic acid, gibberellin, indole-3-acetonitrile, isoquercitrin, isosteviol, jhanol, kaempferol, kaurene, lupeol, luteolin, polystachoside, quercetin, quercitrin, rebaudioside A-F, scopoletin, sterebin A-H, steviol, steviolbioside, steviolmonoside, stevioside, stevioside α-3, stigmasterol, umbelliferone, and xanthophylls.

In the present invention, it is contemplated that Stevia plants per se, that have been merely macerated or pulverized can be used. Alternatively, the compounds within Stevia plants can be extracted with solvents, steam distillation or pressing.

In some preferred embodiments, the compositions of the present invention comprise an extract, which has only been partially purified. In other preferred embodiments, the compositions of the present invention comprise an extract which has been at least partially purified such that one or more of the plant chemicals found in Stevia plants have been isolated. Accordingly, the term “extract” as used herein refers to at least a pulverized Stevia plant or plants. The term “extract” further includes compounds and compositions that have been isolated from the pulverized Stevia plants by solvent extraction, steam distillation, pressing or extraction by an extractor such as Soxhlet extractor.

Examples of solvents usable for extraction include water, alcohols such as methanol, ethanol, propanol and butanol, polyhydric alcohols such as propylene glycol and butylene glycol, ketones such as acetone and methyl ethyl ketone, esters such as methyl acetate and ethyl acetate, linear or cyclic ethers such as tetrahydrofuran and diethyl ether, halogenated hydrocarbons such as dichloromethane, hydrocarbons such as hexane, cyclohexane and petroleum ether, aromatic hydrocarbons such as toluene, polyethers such as polyethylene glycol and pyridines. They may be used either singly or in combination.

There is a variety of suitable extraction methods for extracting compounds from Stevia plants described in the technical literature. For example, Japanese Patent No. 63173531, which issued in 1988 to Nakazato, describes a method of extracting glycosides from Stevia rebaudiana. This method includes the following steps. The first step is to extract a liquid solution from the Stevia rebaudiana plant. Secondly, the liquid solution is passed through a non-polar porous resin, such as amberlite XAD-2 and eluted with a water soluble organic solvent, preferably methanol. Thirdly, the eluted solution is concentrated and dried to give a powdery material. This procedure isolates a mixture of glycosides, but it does not isolate pure single compounds such as Rebaudioside A. Korean Patent No. 9007421 passes the eluted solution from Japanese Patent No. 63173531 through a column which is packed with positive ion-exchange resin (preferably Diaion SK1B) and negative ion-exchange resin (Amberite IRA 904). U.S. Pat. No. 4,892,938, to Giovanetto discloses a purification process in which the aqueous extracts of the Stevia plants are purified by passing these aqueous extracts through a series of ion-exchange resins which are selected to remove various impurities. The glycosides remain in the water and are recovered by evaporation of the water. The advantage is that everything is done in water, while most other processes involve the use of a solvent at some point.

Any or all of the above methods can be used in the present invention to obtain the required Stevia plant extract. Accordingly, all of the above methods for producing an extract from Stevia plants are hereby incorporated in their entirety by reference.

One method of preparing an extract of Stevia plant that is considered particularly useful involves the extraction of a liquid solution from a Stevia plant using room temperature water. The plant solids are then separated followed by the addition of lime and removal of precipitated solids. This initial extraction sequence is common in most of the processes for extracting glycosides.

The extract solution is then passed through an Amberlite XAD-7 resin or reverse phase C18 bonded silica column to remove other materials from the plant. These materials include organic acids and bases, inorganic salts, tannic or phenol like substances, substances derived from the photosynthetic apparatus, proteins and amino acids. The liquid flowing from the resin column contains the compounds of interest. These can either be used directly or further extracted using a water soluble organic solvent such as a methanol solution.

In some preferred compositions of the present invention the compositions comprise at least stevioside and/or stevioside α-3 in purified form admixed with at least one bile salt. A further preferred composition of the present invention consists essentially of stevioside admixed with at least one bile salt.

The term “admixed” and the phrase “in admixture” are synonymous and mean in a state of being in a homogeneous or heterogeneous mixture. It will be appreciated by the skilled reader that the term “admixed” can further refer to the fact that the Stevia extract as defined herein and the at least one bile salt can be simply mixed or blended together by stirring or agitation. However, the term “admixed” can also include other forms of mixing including the chemical reaction of the Stevia extract and the at least one bile salt.

Bile salts are naturally occurring surfactants. They are a group of compounds with a common “backbone” structure based on cholanic acid found in all mammals and higher vegetables. Bile salts may be mono-, di- or tri-hydroxylated; they always contain a 3α-hydroxyl group whereas the other hydroxyl groups, most commonly found at C₆, C₇ or C₁₂, may be positioned either above (β) or below (α) the plane of the molecule.

Within the class of compounds described as bile salts are included amphiphilic polyhydric sterols bearing carboxyl groups as part of the primary side chain. The most common examples of these in mammals result from cholesterol metabolism and are found in the bile and, in derivatised form, throughout the intestine.

Some of the known bile salts, include chenodeoxycholate, cholate, deoxycholate, fusidate, glycholate, glycochenodeoxycholate, glycocholate, glycodeoxycholate, glycolithocholate, glycoursodeoxycholate, lithocholate, taurochenodeoxycholate, taurocholate, taurodeoxycholate, taurodihydrofusidate, taurolithocholate, taurouodeoxycholate and ursodeoxycholate. Each of these compounds can also be functionalised and substituted to encompass a class of compounds, which includes among other things, oxidized and reduced analogs, alkylated and acylated analogs, cyclized or bis-cyclized analogs, and analogs having a shorter or longer side chain. The general structure which contemplates many of these classes of bile acid related compounds is:

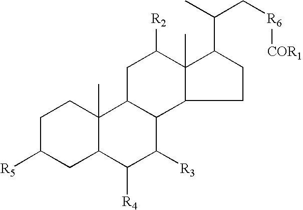

wherein:
R₁, R₂, R₃, R₄, and R₅ are independently hydrogen or XL where
X is nothing, O, S, NH or NL and L is hydrogen, metallic ion, halogen, an alkyl or alenyl radical having up to 10 carbon atoms, which is branched or unbranched, a cycloalkyl radical having 3 to 8 carbon atoms, or a benzyl radical which is unsubstituted or substituted 1 to 3 times by F, Cl, Br, (C₁-C₄)-alkyl or (C₁-C₄)-alkoxy; and where L is bonded to R₁, L can alternatively be an amino acid; and R₆ is (CH₂)ₙ where 0≦n≦5.

Included in these permutations, it is particularly contemplated that R₁ may be amino-, glycine, taurine, alanine or other amino acid group, and R₂, R₃, R₄, and R₅ may independently be amino-, hydroxy-, keto- or halogeno-.

One subclass of compounds specifically contemplated to be effective as active glycoregulatory agents are modified bile acids described in U.S. Pat. No. 5,641,767 to Wess et al., the totality of which is incorporated herein by reference. Still another subclass of compounds specifically contemplated to be effective as active glycoregulatory agents are nor- and homo-bile acid derivatives described in U.S. Pat. No. 5,656,277 to Berlati et al., the totality of which is incorporated herein by reference. Still other subclasses of compounds specifically contemplated to be effective as active glycoregulatory agents are the bile acid derivatives described in U.S. Pat. No. 5,610,151 to Glombik et al., the bile acid derivatives described in U.S. Pat. No. 5,428,182 to Enhsen et al. and the cholerically active esters and salts of bile acids described in U.S. Pat. No. 3,910,888 to Widauer et al. all of which are incorporated herein by reference.

Further examples of suitable bile salts include salts (e.g., sodium or potassium salts) of fatty acids such as cholic acid, chenodeoxycholic acid, glycocholic acid, taurocholic acid, glycochenodeoxycholic acid, taurochenodeoxycholic acid, deoxycholic acid, glycodeoxycholic acid, taurodeoxycholic acid, lithocholic acid, and ursodeoxycholic acid. Preferred are the trihydroxy bile salts, such as the salts (e.g., potassium and sodium salts) of cholic, glycocholic and taurocholic acids. Particularly preferred are sodium taurocholate and potassium taurocholate.

In the context of this specification, the term “bile salt” may also apply to synthetic analogues of naturally occurring bile salts which display similar biological effects, or to microbially derived molecules such as fusidic acid and its derivatives.

The bile salt (or salts) may be either unconjugated or conjugated. The term “unconjugated” refers to a bile salt in which the primary side chain has a single carboxyl group which is at the terminal position and which is unsubstituted. Examples of unconjugated bile salts include cholate, ursodeoxycholate, chenodeoxycholate and deoxycholate. A conjugated bile salt is one in which the primary side chain has a carboxyl group which is substituted. Often the substituent will be an amino acid derivative which is linked via its nitrogen atom to the carboxyl group of the bile salt. Examples of conjugated bile salts include taurocholate, glycocholate, taurodeoxycholate and glycodeoxycholate.

Thus, in the present invention the term “bile salts” includes any or all of the above mentioned bile salts, salt thereof or derivative thereof.

In one especially preferred embodiment, the compositions of the present invention comprises or consist essentially of the bile salt 3α,7α-dihydroxy-12-oxo-5β-cholanate (12-monoketocholanate) and/or salts of: dehydrocholic acid, 7,12 diketocholic acid, 3,7 diketochoic acid, 7-monoketocholic acid.

Bile salts and acids according to the present inventive subject matter can be readily synthesised according to known chemistry. In several instances, such as deoxycholic acid, cholic acid, taurocholic acid, glycocholic acid, glycodeoxycholic acid, taurodeoxycholic acid, ursodeoxycholic acid and chenodeoxycholic acid, the bile salts are commercially available in purified forms. In other instances, such as the various modified bile acids and analogs thereof discussed above, the compounds can be synthesized according to procedures set forth or readily derivable from the various identified patents.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, butyl-, tetramethylammonium salts and the like.

Also intended as pharmaceutically acceptable acid addition salts are the hydrates, which the present compounds, are able to form.

Furthermore, the pharmaceutically acceptable salts comprise basic amino acid salts such as lysine, arginine and ornithine.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

In one preferred embodiment, the composition of the present invention consists essentially of stevioside and 3α,7α-dihydroxy-12-oxo-5β-cholanate.

Once the bile salt(s) and Stevia extract are admixed they can be used directly to treat or prevent hyperglycaemic conditions or alternatively, they can be combined with other carriers, adjuvants or diluents to formularise the compositions for administration.

It is contemplated that any suitable membrane can be used to receive a cholerically active composition of the present invention to modulate blood lipid levels. In addition to nasal membranes, for example, compositions according to the present inventive subject matter can be applied rectally, vaginally, orally, (sublingually or bucally), conjunctively, or by inhalation. It is also contemplated that compositions according to the present inventive subject matter can even be effective when absorbed through the mucous membranes of the digestive tract.

As such, compositions according to the present inventive subject matter may be provided in virtually any state, including a liquid, e.g. adapted for administration as a spray, a gel, or even a solid, eg. a powder acceptable for snuffing. Such preparations will usually include ancillary agents, for example a pH-buffering system, preferably a buffer such as phosphate, citrate or acetate buffers, a preservative and an osmotic pressure controlling agent, eg. glycerol or sodium chloride. Powder formulations may contain in addition to the compositions of the present invention, an acceptable powdery diluent or mixture thereof, such as cellulose or derivatives thereof, for example cellulose ethers or sodium carboxymethylcellulose, starch, a long chain fatty acid or a salt thereof, eg. aluminium stearate, an organic polymer, eg. of an acrylic acid derivative or inorganic vehicles, such as talc or diatomaceous earth. Supplementary addition of water-absorbing polymers, for example polyethylene glycol or polyvinyl pyrrolidone may be desirable to improve adhesion of the powder formulation to the nasal or other mucosa.

Preferred liquid preparations are those in which the diluent is water. Such preparations may be prepared by dispersing the absorption enhancing system in the aqueous medium containing the compositions of the present invention and ancillary agents, the dispersion being conducted by any method usually employed for suspension or emulsification, eg. ultrasonic treatment. Adjustment of the aqueous phase to neutrality (i.e. to pH in the range from about 6.5 to about 8) may be accomplished in any of the preparatory steps. Preferably, microemulsions are prepared in which the size of the dispersed particles or droplets is of the order of 10 nm, thereby facilitating their passage across the mucosa. Such microemulsions may be sterilized by filtration. Where a phospholipid or fatty oil is included in the formulations, such additive may advantageously be present in the range of from 0.01 to 10%, preferably from 0.5 to 5% (w/v), and 0.01-50%, preferably from 0.1 to 10% (w/v), respectively, of the preparation. Due to the fact that proteases and peptidases are associated with the nasal mucosa (see Stratford & Lee, Int. Journ. Pharmaceutics, 30: 73-82, 1986), it may be desirable to incorporate biocompatible protease and peptidase inhibitors into polypeptide containing formulations.

Suitable carriers for use in the present invention include, but are not limited to, pyrogen-free saline. For parenteral administration of the compositions, a sterile solution or suspension is prepared in saline that may contain additives, such as ethyl oleate or isopropyl myristate, and can be injected, for example, into subcutaneous or intramuscular tissues.

Suitable carriers for oral administration of compositions can include one or more substances which may also act as flavouring agents, lubricants, suspending agents, or as protectants. Suitable solid carriers include calcium phosphate, calcium carbonate, magnesium stearate, sugars, starch, gelatine, cellulose, carboxypolymethylene, or cyclodextrans. Suitable liquid carriers may be water, pharmaceutically accepted oils, or a mixture of both. The liquid can also contain other suitable pharmaceutical additions such as buffers, preservatives, flavouring agents, viscosity or osmo-regulators, stabilizers or suspending agents. Examples of suitable liquid carriers include water with or without various additives, including carboxypolymethylene as a pH-regulated gel. The compositions may be contained in enteric coated capsules that release the compositions into the intestine to avoid gastric breakdown.

Alternatively, the compositions may be microencapsulated with either a natural or a synthetic polymer into microparticles 4-8 μm in diameter, which target intestinal lymphoid tissues and produce a sustained release of compounds for up to four weeks.

It is contemplated that the various compositions of the present invention may be administered trans-mucosally in any suitable dosage, and according to any suitable regime depending upon the subjects weight, the severity of the symptoms being treated, the amount of composition desired to be absorbed, and the experience and judgment of the prescribing professional. Generally, the appropriate dosage will be that which properly balances the intended results against toxicity and other side effects. Where the condition being treated involves hyperlipidemia, for example, an amount is preferred that decreases blood cholesterol to a normal or near normal ranges. Also preferred is an amount that causes a sustained reduction in blood cholesterol levels.

In a further embodiment of the invention the present compositions may be administered in combination with one or more pharmacologically active substances eg selected from anti-diabetics, anti-obesity agents, anti-hypertensive agents and agents for the treatment and/or prevention of complications resulting from or associated with diabetes.

Suitable anti-diabetics comprise insulin, GLP-1 derivatives such as those disclosed in WO98/08871 to Novo Nordisk A/S, which is incorporated herein by reference, as well as orally active hypoglycaemic agents.

The orally active hypoglycaemic agents preferably comprise sulphonylureas, biguanides, meglitinides, oxadiazolidinediones, thiazolidinediones, glucosidase inhibitors, glucagon antagonists, GLP-1 agonists, potassium channel openers such as those disclosed in WO97/26265 and WO99/03861 to Novo Nordisk A/S which are incorporated herein by reference, insulin sensitizers, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as anti-hyperlipidemic agents and anti-lipidemic agents, compounds lowering food intake, PPAR (peroxisome proliferator-activated receptor) and RXR (retinoid X receptor) agonists and agents acting on the ATP-dependent potassium channel of the β-cells.

In some embodiments of the present invention the compositions are administered in combination with insulin.

In further embodiments the compositions are administered in combination with a sulphonylurea eg tolbutamide, glibenclamide, glipizide or glicazide.

In other embodiments the compositions are administered in combination with a biguanide eg metformin.

In yet other embodiments the compositions are administered in combination with a meglitinide eg repaglinide.

In still other embodiments the compositions are administered in combination with a thiazolidinedione eg troglitazone, ciglitazone, pioglitazone, rosiglitazone or the compounds disclosed in WO97/41097 to Dr. Reddy's Research Foundation.

Furthermore, the present compositions may be administered in combination with the insulin sensitizers disclosed in WO99/19313 to Dr. Reddy's Research Foundation.

In further embodiments the compositions are administered in combination with an α-glucosidase inhibitor eg miglitol or acarbose.

In other embodiments the compositions are administered in combination with an agent acting on the ATP-dependent potassium channel of the β-cells eg tolbutamide, glibenclamide, glipizide, glicazide or repaglinide.

Furthermore, the present compositions may be administered in combination with nateglinide. In still another embodiment the present compounds are administered in combination with an anti-hyperlipidemic agent or anti-lipidemic agent eg cholestyramine, colestipol, clofibrate, gemfibrozil, lovastatin, pravastatin, simvastatin, probucol or dextrothyroxine.

In further embodiments the present compositions are administered in combination with more than one of the above-mentioned compounds eg in combination with a sulphonylurea and metformin, a sulphonylurea and acarbose, repaglinide and metformin, insulin and a sulphonylurea, insulin and metformin, insulin and troglitazone, insulin and lovastatin, etc.

Furthermore, the compositions according to the invention may be administered in combination with one or more anti-obesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators or TRp agonists.

In some embodiments of the invention the anti-obesity agent is leptin.

In other embodiments the anti-obesity agent is dexamphetamine or amphetamine.

In other embodiments the anti-obesity agent is fenfluramine or dexfenfluramine.

In still other embodiments the anti-obesity agent is sibutramine.

In further embodiments the anti-obesity agent is orlistat.

In other embodiments the anti-obesity agent is mazindol or phentermine.

Furthermore, the present compositions may be administered in combination with one or more anti-hypertensive agents. Examples of anti-hypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compositions according to the invention with one or more of the above-mentioned compounds and optionally one or more pharmacologically active substances are considered to be within the scope of the present invention.

Once an appropriate formulation of the compositions of the inventions has been determined considering route of administration, age, sex, weight and the like of the subject to be treated it is administrated as described supra.

The terms “subject” or “individual” are used interchangeably-herein to refer to any member of the class mammalia, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs. The terms do not denote a particular age. Thus, both adult and newborn individuals are intended to be covered.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses.

Generally, the terms “treating,” “treatment” and the like are used herein to mean affecting an individual or subject, their tissue or cells to obtain a desired pharmacological and/or physiological effect. The effect may be prophylactic in terms of completely or partially preventing the hyperlipidemia or sign or symptom thereof, and/or may be therapeutic in terms of a partial or complete cure of the hyperlipidemia. “Treating” as used herein covers any treatment of, or prevention of hyperlipidemia in a mammal, particularly a human, and includes: (a) preventing hyperlipidemia from occurring in a subject that may be predisposed to hyperlipidemia, but has not yet been diagnosed as having it; (b) inhibiting hyperlipidemia, i.e., arresting its development; or (c) relieving or ameliorating the symptoms of hyperlipidemia, i.e., cause regression of the symptoms of hyperlipidemia.

The terms “effective amount” or “therapeutically effective amount” refers to that amount which is sufficient to treat, reduce, inhibit or prevent hyperlipidemia in a subject. Equally, the term “effective amount” when used with reference to a composition's anti-hyperlipidemia activity means the amount sufficient to reduce or inhibit hyperlipidemia in a subject. What constitutes an effective amount, or dose, of a composition of the invention depends, among other factors, on the body weight of the subject and the reduction in hyperlipidemia required. Normally an effective dose will be found in the range of about 1 to about 6 mg/kg body weight, more preferably, the average oral daily dosage would be about 2 mg/kg of body mass, which equates to about 140 mg/day for a 70 kg person. Proportionately smaller or larger doses can be appropriate for subjects having lesser or greater body weight. Such a dose can be administered as needed, but typically administration 1 to about 4 times per day, in most cases 1 or 2 times a day, provides an adequate reduction in hyperlipidemia.

In some instance, the methods of the invention are provided in use. For example, use of a composition comprising an extract from at least one plant from the genus Stevia and at least one bile salt, salt thereof or derivative thereof for the treatment of hyperlipidemia.

By “comprising” is meant including, but not limited to, whatever follows the word comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Further details of practicing this inventive subject matter are furnished by way of the following examples which, however, should not be construed so as to imposes any kind of limitation to the scope of the invention.

### Example 1 Composition Comprising Bile Salt and Steviozide

40 mg of the sodium salt of 3α,7α-dihydroxy-12-oxo-5β-cholanate (representive bile salt) was admixed with 200 mg of stevioside in 0.9% sodium chloride solution and sonicated at 100 Wats for 30 seconds at 37° C.

### Example 2 Treatment of Hyperglycaemia

In this study white, adult male Wistar rats with average weights between 200 to 300 grams were used. During the study, the animals had free access to food and water and were exposed to changes of dark and light every 12 hours. Access to the food was restricted 12 hours before measurement of glucose blood level with free access to water.

The experiment was performed on a normoglycaemic group (Group 1, 32 rats, 8 per treatment) and diabetic rats (Group 2, 32 rats, 8 per treatment). For the diabetic rat experiments diabetes was induced with intraperitoneal injection of alloxan 100 mg/kg. Alloxan is very well verified diabetogenic substance for inducing diabetes in rats and other animal species (rabbit, dog, mice et cetera). See, for example, McLetchie, J.R. Coil Physicians Edinb, 32: 134-142, 2002; Lenzen & Panten, Diabetologia, 31: 337-342, 1988; Shaw et al., Lancet, 11: 384-386, 1943; Goldner & Gomori, Endocrinology, 35: 241-268, 1943.

The treatments were:

1). Saline solution, 1 ml/kg, per orally, daily for 5 days (control group);
2). Stevioside, 20 mg/kg, orally, daily for 5 days;
3). Sodium salt of 3α,7α-dihydroxy-12-oxo-5β-cholanate (monoketocholic acid-MKC), 4 mg/kg, orally, daily for 5 days; and
4). 1 ml/kg of the composition described in Example 1, orally, daily for 5 days.

In the diabetic rat the criterion for successful diabetes induction was blood glucose greater than 20 mmol/L on the third day after the last alloxan dose after 3 hours of fasting with free access to water.

15 minutes after the 5^{th} dose, the animals were exposed to an oral glucose tolerance test (OGTT) using 4 g/kg, orally. Glucose blood concentrations were measured before the treatment, after the 5^{th} dose, before OGTT, and 30 minutes after OGTT. Then the animals were anaesthetised with urethane injected intraperitoneally, 750 mg/kg, and blood samples for further biochemical analyses were taken by cardiopunction.

All biochemical analyses of serum were done by standard procedures, as recommended by the International Federation for Clinical Chemistry (IFCC), on automatic spectrophotometer LISA 300 plus with International Quality Control. The following were measured in serum:

1). Triglycerides concentration (GPO-PAP kit obtained from Boehringer Mannheim GmbH (Mannheim, Germany) in accordance with the manufacturers instructions)
2). Total low density lipoprotein (LDL) and high density lipoprotein (HDL)
3). Cholesterol concentration (CHOD-PAP kit obtained from Boehringer Mannheim GmbH (Mannheim, Germany) in accordance with the manufacturers instructions); and
4). C-peptide concentration.

As shown in Table 1 in the non-diabetic rats the combination of stevioside and 3α,7α-dihydroxy-12-oxo-5β-cholanate significantly decreased glucose levels after the OGTT in normal rats, but no treatment significantly decreased glucose levels before the OGTT.

In contrast, in the diabetic rats receiving only 3α,7α-dihydroxy-12-oxo-5β-cholanate there was a significant decrease in glucose levels both before and after the OGTT, while the group receiving the composition of the present invention showed a highly significant reduction in glucose levels before and after the OGTT.

This means that in diabetics the use of stevioside plus 3α,7α-dihydroxy-12-oxo-5β-cholanate was an effective method of reducing glucose levels before and after glucose loading. It can reasonably be expected that at increased doses of 3α,7α-dihydroxy-12-oxo-5β-cholanate and stevioside would lead to larger reductions. 3α,7α-dihydroxy-12-oxo-5β-cholanate and stevioside alone also conferred beneficial effects on glucose levels under increased glucose loading during the OGTT.

TABLE 1

| | | | |
|---|---|---|---|
| Group & Treatment | Before | After treatment (one | |
| (n = 8) | treatment | dose daily for 5 days) | After OGTT |

Glucose blood concentration in normoglycaemic Wistar rats (mmol/L)

| | | | |
|---|---|---|---|
| Control (saline) | 6.1 ± 1.06 | 6.12 ± 0.92 | 19.4 ± 5.06 |
| MKH (4 mg/kg orally | 6.82 ± 2.15 | 5.42 ± 1.15 | 8.52 ± 3.19** |
| per day) | | | |
| Stevioside (20 mg/kg | 6.95 ± 0.99 | 5.47 ± 0.67 | 11.4 ± 2.9** |
| orally per day) | | | |
| MKH (4 mg/kg orally | 6.73 ± 1.48 | 4.88 ± 0.4 | 8.5 ± 4.7** |
| per day) plus | | | |
| Stevioside (20 mg/kg | | | |
| orally day) | | | |

Glucose blood concentration in diabetic Wistar rats (mmol/L)

| | | | |
|---|---|---|---|
| Control (saline) | 25.42 ± 2.21 | 25.27 ± 1.92 | 34.72 ± 5.06** |
| MKH (4 mg/kg orally | 24.73 ± 2.15 | 22.34 ± 1.15 x | 27.05 ± 1.17* |
| per day) | | | |
| Stevioside (20 mg/kg | 25.14 ± 1.87 | 22.53 ± 1.07* | 28.03 ± 1.08* |
| orally per day) | | | |
| MKH (4 mg/kg orally | 26.12 ± 2.01 | 21.03 ± 0.81** x | 26.36 ± 1.02** |
| per day) plus | | | |
| Stevioside (20 mg/kg | | | |
| orally day) | | | |

*significance p < 0.05 in comparison to vertical control group for each treatment
**significance p < 0.01 in comparison to vertical control for each treatment
x significance p < 0.05 in comparison to horizontal control group before treatment

Endogenous C-peptide measurement is the most accepted measurement for level of endogenous insulin secretion as it is co-secreted with insulin at a 1:1 molar ratio. It is also the best primary outcome for clinical trials of therapies aimed at preserving or improving beta cell function and endogenous insulin secretion in type 1 diabetics (see, for example, Palmer et al. Diabetes, 53: 250-264, 2004).

Treatment of diabetic rats with Stevioside alone or Stevioside plus 3α,7α-dihydroxy-12-oxo-5β-cholanate for 5 days resulted in significant increases in C-peptide (see Table 2). This same treatment resulted in a reduction in glucose levels from the elevated levels present in the diabetic rats (see Table 1), although the reduced glucose levels were still higher than those of the control non-diabetic rats. These data show that the combination of Stevioside plus 3α,7α-dihydroxy-12-oxo-5β-cholanate is an effective treatment for increasing endogenous insulin secretion in Type 1 and 2 diabetics.

Neither stevioside or 3α,7α-dihydroxy-12-oxo-5β-cholanate alone had statistically significant effects on C peptide levels in non-diabetic rats. Interestingly the effect of the stevioside and 3α,7α-dihydroxy-12-oxo-5β-cholanate combined-treatment was to reduce C peptide levels below those of the control treatment rats. This was most likely due to the highly significant decreases in glucose levels seen in the stevioside plus 3α,7α-dihydroxy-12-oxo-5β-cholanate treated rats (see Table 1) resulting in a decreased demand for insulin.

TABLE 2
EFFECT ON C-PEPTIDE LEVELS IN DIABETIC
AND NON-DIABETIC RATS OF TREATMENTS¹

| | |
|---|---|
| Group (n = 8) | C peptide (pmol/l) |

C peptide serum concentration (pmol/l)
in normoglycaemic rats

| | |
|---|---|
| Control | 313.25 ± 18.36 |
| Stevioside (20 mg/kg orally per day) | 294 ± 93.2 |
| MKH (4 mg/kg orally per day) | 230.87 ± 80.64 |
| MKH (4 mg/kg orally per day) plus | 214.75 ± 79.04 ** |
| Stevioside (20 mg/kg orally per day) | |

C peptide serum concentration (pmol/l) in diabetic rats

| | |
|---|---|
| Diabetic Control | 167.82 ± 18.36 |
| Stevioside (20 mg/kg orally per day) | 230.12 ± 28.45 * |
| MKH (4 mg/kg orally per day) | 178.38 ± 34.25 |
| MKH (4 mg/kg orally per day) plus | 214.75 ± 20.12 * |
| Stevioside (20 mg/kg orally per day) | |

¹ Treatments are as described for Table 1. Treatment doses were administered daily for 5 days.
* significance p < 0.05
** significance p < 0.01

### Example 3 Effect on Cholesterol and Serum Lipid Profiles

The composition from Example 1 was trialled to see the effect on cholesterol levels and on serum lipid profiles. Generally the desired outcome was a decrease in low density lipoprotein (LDL), with no concomitant decrease in high density lipoprotein (HDL). It was also hypothesised that there would be a compatibility with glycaemic effects shown in Example 2.

It will be appreciated by those skilled in the field that Type 1, and Type 2 diabetics have increased risks of cardiovascular diseases due to serum lipid profiles. Current therapies such statins have side effects and do not work effectively for subjects homozygous for familial hypercholesterolemia.

The experimental procedures were essentially those described in Example 2.

Table 3 shows that there were no significant changes in total cholesterol in serum concentrations after MKH and stevioside treatments. Table 4 shows that there were no significant changes in HDL serum cholesterol concentrations after MKH and stevioside treatments.

TABLE 3
Serum total cholesterol conc. (mmol/l) in normoglycaemic rats

| Group (n = 8) | |
|---|---|
| Control | 1.33 ± 0.47 |
| Stevioside (20 mg/kg orally per day) | 1.1 ± 0.30 |
| MKH (4 mg/kg orally per day) | 0.99 ± 0.30 |
| MKH (4 mg/kg orally per day) plus | 1.22 ± 0.31 |
| Stevioside (20 mg/kg orally per day) | |

TABLE 4
HDL serum cholesterol concentrations(mmol/l)
in normoglycaemic rats

| Group (n = 8) | |
|---|---|
| Control | 0.92 ± 0.35 |
| Stevioside (20 mg/kg orally per day) | 0.78 ± 0.18 |
| MKH (4 mg/kg orally per day) | 0.73 ± 0.20 |
| MKH (4 mg/kg orally per day) plus | 0.79 ± 0.27 |
| Stevioside (20 mg/kg orally per day) | |

In Table 5 it can be seen that LDL serum cholesterol concentrations were significantly decreased (one third of control values) after stevioside treatment.

LDL serum cholesterol concentrations were significantly decreased (one third of control values) after 5 days of MKH treatment.

LDL serum cholesterol concentrations were significantly decreased (one third of control values) after 5 days of MKH plus Stevioside treatment.

As shown in Table 6, there were no significant changes in total cholesterol in serum concentrations of diabetic or non-diabetic rats after MKH, Stevioside, and MKH plus Stevioside treatments.

TABLE 5
LDL serum cholesterol concentrations
(mmol/l) in normoglycaemic rats

| Group | |
|---|---|
| Control | 0.38 ± 0.17 |
| Stevioside (20 mg/kg orally per day) | 0.1 ± 0.08 ** |
| MKH (4 mg/kg orally per day) | 0.11 ± 0.04 ** |
| MKH (4 mg/kg orally per day) plus | 0.125 ± 0.05 ** |
| Stevioside (20 mg/kg orally per day) | |

TABLE 6
Serum total cholesterol concentrations (mmol/l) in normoglycaemic
and diabetic rats after 5 days treatment

| | Normoglycaemic | Diabetic |
|---|---|---|
| Group | (n = 8) | (n = 8) |
| Control | 1.33 ± 0.47 | 1.44 ± 0.19 |
| Stevioside (20 mg/kg orally per day) | 1.1 ± 0.30 | 1.39 ± 0.31 |
| MKH (4 mg/kg orally per day) | 0.99 ± 0.30 | 1.03 ± 0.26 |
| MKH (4 mg/kg orally per day) plus | 1.22 ± 0.31 | 1.37 ± 0.03 |
| Stevioside (20 mg/kg orally per day) | | |

In non-diabetic rats LDL serum cholesterol concentrations were significantly decreased (one third of control values) after stevioside treatment. LDL serum cholesterol concentrations were significantly decreased (one third of control values) after 5 days of MKH treatment. LDL serum cholesterol concentrations were significantly decreased (one third of control values) after 5 days of combined treatment with stevioside plus MKH.

In diabetic rats the stevioside treatment for 5 days resulted in a highly significant decrease in LDL cholesterol levels. Inventors note that the MKH and MKH+stevioside treatments resulted in decreases in LDL levels and while these were not statistically significant data in Tables 7 to 9. Table 10 shows that with a longer treatment period (7 days) MKH alone resulted in a very highly significant decrease in LDL levels in diabetic rats.

Decreases in LDL levels are a valuable therapeutic outcome as high LDL levels are strongly associated with increased levels of cardiovascular events and atherosclerotic risk. It has been shown that a 10% increase in LDL cholesterol levels was associated with a 15% increase in ischemic heart disease. In diabetic patients a consequence of increases in LDL levels and decreased HDL levels frequently associated with Type 1 and 2 diabetes is increased risk of atherosclerosis. Note the elevated levels of LDLs in control diabetic rats versus non-diabetic rats in this study.

TABLE 7
LDL cholesterol serum concentrations (mmol/l) in normoglycaemic
and diabetic rats after 5 days treatment

| | Normoglycaemic | |
|---|---|---|
| Group (N = 8) | (n = 8) | Diabetic (n = 8) |
| Control | 0.38 ± 0.17 | 0.63 ± 0.06 |
| Stevioside (20 mg/kg orally | 0.1 ± 0.08 ** | 0.42 ± 0.09 ** |
| per day) | | |
| MKH (4 mg/kg orally per day) | 0.11 ± 0.04 ** | 0.56 ± 0.17 |
| MKH (4 mg/kg orally per day) | 0.125 ± 0.05 ** | 0.48 ± 0.09 |
| plus Stevioside (20 mg/kg | | |
| orally per day) | | |

TABLE 8
HDL cholesterol serum concentrations (mmol/l) in normoglycaemic
and diabetic rats after 5 days treatment

| | Normoglycaemic | |
|---|---|---|
| Group | (n = 8) | Diabetic (n = 8) |
| Control | 0.92 ± 0.35 | 0.69 ± 0.16 |
| Stevioside (20 mg/kg orally | 0.78 ± 0.18 | 0.85 ± 0.20 |
| per day) | | |
| MKH (4 mg/kg orally per day) | 0.73 ± 0.20 | 0.50 ± 0.12 !! |
| MKH (4 mg/kg orally per day) | 0.79 ± 0.27 | 0.80 ± 0.01 x |
| plus Stevioside (20 mg/kg | | |
| orally per day) | | |

Decreases in HDL levels are associated with increased risk of coronary heart disease. No decreases in HDL were observed in any treatment of non-diabetic rats. A decrease was observed in diabetic rats treated with MKH at 4 mg/kg for 5 days although this effect was not observed in diabetic rats treated for 7 days with MKH at 2 mg/kg. Treatment with MKH+stevioside resulted in a statistically significant increase in beneficial HDL levels.

TABLE 9
LDL: HDL Ratio in normoglycaemic and diabetic
rats (ratios of means) after 5 days treatment

| | Normoglycaemic | Diabetic |
|---|---|---|
| Group (n = 8) | (n = 8) | (n = 8) |
| Control | 0.41 | 0.91 |
| Stevioside (20 mg/kg orally per day) | 0.13 | 0.49 |
| MKH (4 mg/kg orally per day) | 0.01 | 1.12 |
| MKH (4 mg/kg orally per day) plus | 0.16 | 0.60 |
| Stevioside (20 mg/kg orally per day) | | |

Decreased LDL levels and increased HDL levels (low LDL:HDL ratios) are a desired outcome of therapies to modulate serum lipid levels. In non-diabetic rats all three treatments gave large decreases in LDL:HDL levels. In diabetic rats decreased ratios were also observed with the exception of MKH alone which showed a slight increase in LDL:HDL ratio. We note that by Day 7 the MKH treatment did result in a significant decrease in the LDL:HDL ratio (Table 10) due to a slight increase in HDL levels and a large decrease in LDL levels in diabetic rats.

TABLE 10
Serum cholesterol concentrations after 7 day treatment
of diabetic rats orally with saline, lovastatin 20 mg/kg
and MKH 2 mg/kg and Lovastatin and MKH combination

| | Control group | | | Lovastatin |
|---|---|---|---|---|
| | Saline | | | (20 mg/kg) |
| | solution | Lovastatin | MKH | Plus MKH |
| Parameters | (20 ml/kg) | (20 mg/kg/day) | (2 mg/kg/day) | (2 mg/kg/day) |
| (Diabetic rats n = 8) | orally | orally | orally | orally |
| Total cholesterol | 1.93 ± 0.45 | 1.19 ± 0.06 | 1.21 ± 0.14 | 1.22 ± 0.04 |
| (mmol/l) | | | | |
| HDL cholesterol | 1.08 ± 0.13 | 0.74 ± 0.16 | 0.89 ± 0.14 | 0.82 ± 0.06 |
| (mmol/l) | | | | |
| LDL cholesterol | 0.49 ± 0.31 | 0.09 ± 0.17 | 0.03 ± 0.04 | 0.12 ± 0.09 |
| (mmol/l) | | | | |
| LDL/HDL ratio | 0.45 ± 0.28 | 0.15 ± 0.29 | 0.04 ± 0.04 | 0.15 ± 0.18 |

Oral dosage of MKH, Lovastatin and Lovastatin plus MKH reduced total cholesterol by a similar amount. However while Lovastatin reduced HDLs significantly MKH did not do so. This is significant as HDLs are protective against heart disease (Despres et al., 2000, Atherosclerosis 153: 263-272). Additionally, the reduction of LDLs with an oral dose of MKH was significantly greater than that achieved by Lovastatin. This is important as low LDL levels are associated with decreased risk of heart disease. The dose of MKH used in this study was orally administered showing that this administration route is effective and the dose rate is lower than used in other experiments (see above) and it is expected that the magnitude of the beneficial effects shown in Table 10 can be increased by a larger dose if required.

1. A composition comprising an extract from at least one plant from the genus Stevia and at least one bile salt, salt thereof or derivative thereof admixed in a form suitable for therapeutic administration.

2. The composition of claim 1, wherein said extract is isolated from a plant selected from the group consisting of Stevia lemmonii, Stevia micrantha, Stevia ovata, Stevia plummerae, Stevia rebaudiana, Stevia salicifolia, Stevia serrata and Stevia viscida.

3. The composition of claim 1, wherein said extract is isolated from Stevia rebaudiana.

4. The composition of claim 1, wherein said extract comprise at least one compound selected from the group consisting of apigenin, austroinulin, avicularin, beta-sitosterol, caffeic acid, campesterol, caryophyllene, centaureidin, chlorogenic acid, chlorophyll, cosmosiin, cynaroside, daucosterol, diterpene glycosides, dulcosides A-B, foeniculin, formic acid, gibberellic acid, gibberellin, indole-3-acetonitrile, isoquercitrin, isosteviol, jhanol, kaempferol, kaurene, lupeol, luteolin, polystachoside, quercetin, quercitrin, rebaudioside A-F, scopoletin, sterebin A-H, steviol, steviolbioside, steviolmonoside, stevioside, stevioside cc-3, stigmasterol, umbelliferone, and xanthophylls.

5. The composition of claim 1, wherein the extract comprises at least one of steviolbioside, steviolmonoside, stevioside, stevioside α-3 and stigmasterol .

6. The composition of claim 1, wherein the extract comprises at least stevioside.

7. The composition of claim 1, wherein the at least one bile salt is selected from the group consisting of chenodeoxycholate, cholate, deoxycholate, fusidate, glycholate, glycochenodeoxycholate, glycocholate, glycodeoxycholate, glycolithocholate, glycoursodeoxycholate, lithocholate, taurochenodeoxycholate, taurocholate, taurodeoxycholate, taurodihydrofusidate, taurolithocholate, taurouodeoxycholate and ursodeoxycholate.

8. The composition of claim 1, wherein the at least one bile salt is selected from the group consisting of 3α,6α-Dihydroxy-5β-cholanic acid, 3α, 6α,7α-Trihydroxy-5β-cholanic acid, 3α,7β-Dihydroxy-5β-cholanic acid, 3α,7α-Dihydroxy-24-ethyl-5β-cholestan-26-carboxylic acid, 2β,3α,7α,12α-Tetrahydroxy-5β-cholestan-24-carboxylic acid, 1β,3α,7α,12α-Tetrahydroxy-5β-cholestan-24-carboxylic acid, 3α,7β,12α-Trihydroxy-5β-cholestan-26-carboxylic acid, 3α,7α-Dihydroxy-7-keto-5β-cholestan-26-carboxylic acid, 3α,7α-Dihidroksy-5β-cholestan-26-carboxylic acid, 3α,12α,22-Trihydroxy-5β-cholestan-26-karboxylic acid, 3α,7α,12α,26-Tetrahydroxy-5β-cholestan-23-en-27-carboxylic acid, 3α,7α, 12α,24-Tetrahydroxy-24-methyl-5β-cholestan-26-carboxylic acid, and 3α,7α,12α-Trihydroxy-26, 27-dinor-5β-cholestan-25-carboxylic acid.

9. A method for the treatment or prevention of hyperlipidemia in a mammalian subject comprising the administration of a therapeutically effective amount of the composition of claim 1.

10. The method of claim 9, wherein said administration is orally.

11. The method of claim 9, wherein said mammalian subject is a dog, a cat, a livestock animal, a horse, or a primate.

12. The method of claim 11, wherein the primate is a human.

13. A method for the treatment or prevention of disorders or diseases mediated by hyperlipidemia, said method comprising administering to a mammalian subject in need thereof an effective amount of a composition comprising an extract from at least one plant from the genus Stevia and at least one bile salt, salt thereof or derivative thereof admixed in a form suitable for therapeutic administration.

14-17. (canceled)

18. The method of claim 13 in which the disease associated with dyslipidemia is selected from the group consisting of coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp (a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.
